(19) United States
(12) Patent Application Publication
(10) Pub. No.: US 2006/0045847 A1
(43) Pub. Date: Mar. 2, 2006

(54) Method for determination of anabolic activity

(76) Inventors: Fernand Labrie, Ste-Foy (CA); Jean Morisette, Ste-Foy (CA); Van Luu-The, Charny (CA); Ezequiel Luis Calvo, Ste-Foy (CA); Guy Bellemare, Quebec (CA)

Correspondence Address: OSTROLENK FABER GERB & SOFFEN, 1180 AVENUE OF THE AMERICAS, NEW YORK, NY, 100368403, US

(21) Appl. No.: 11/214,517

(22) Filed: Aug. 30, 2005

Related U.S. Application Data
(60) Provisional application No. 60/606,174, filed on Aug. 30, 2004.

(51) Int. Cl. A61K 49/00; C12Q 1/68; G06F 19/00

(52) U.S. Cl. 424/9.2; 435/6; 702/20

(57) ABSTRACT

Novel methods for determining the anabolic activity of a compound in muscle using microarrays to compare the in vivo changes of the genomic profile of mammalian muscle induced by a tested compound versus the corresponding changes induced by a known anabolic steroid. For example, in vivo changes of the genomic profile in the mouse induced by a tested compound may be compared to the genomic profile changes induced by the androgenic and anabolic steroid dihydrotestosterone (DHT).

### CROSS-REFERENCE TO RELATED APPLICATION(S)

The present application claims priority of U.S. Provisional Application Ser. No. 60/606,174 filed Aug. 30, 2004.

### FIELD OF THE INVENTION

The present invention relates to a method for determining the anabolic or androgenic activity of a compound in muscles using microarrays. In particular, the present invention compares the in vivo changes induced by such a compound on the genomic profile of a mammalian animal versus the corresponding genomic profile induced by a known anabolic or androgenic steroid.

### BACKGROUND OF THE RELATED ART

Identification of tetrahydrogestrinone (THG) by the US Anti-Doping Agency has created a wave of shock in the world of elite athletes, coaches and suppliers of performance-enhancing drugs [Knight, J., Drugs bust reveals athletes' secret steroid. Nature, 2003. 425(6960): p. 752.]. Soon after development of the appropriate test, THG has been found in a growing list of Olympic medal winners and other prestigious athletes. Being, after norbolethone [Catlin, D. H., B. D. Ahrens, and Y. Kucherova, Detection of norbolethone, an anabolic steroid never marketed, in athletes' urine. Rapid Commun Mass Spectrom, 2002. 16(13): p. 1273-5], the second doping drug never marketed as a pharmaceutical, the in vivo biological and toxicological activities of THG are unknown, thus increasing the risks of its human use.

THG has been a large drug scandal, [Kondro, W., Athletes' “designer steroid” leads to widening scandal. Lancet, 2003. 362(9394): p. 1466]. The first drug tests by the International Olympic Committee were run at the 1968 Olympic Games in Mexico where only ethanol was found, while the detection of the first banned drugs was first made in six olympic athletes in Munich in 1972. THG may escape detection since the compound degrades during standard gas chromatography and mass spectrometry procedures [Kondro, W., Athletes' “designer steroid” leads to widening scandal. Lancet, 2003. 362(9394): p. 1466]. THG has been identified in the laboratory of Donald Catlin at UCLA in June 2003 from a sample sent in a syringe to the US Anti-Doping Agency [Knight, J., Drugs in sport: no dope. Nature, 2003. 426(6963): p. 114-5]. THG differs from gestrinone by reduction of the ethynyl to an ethyl group at position 17α. The present data show that this steroid has 20% the activity of DHT, the most potent natural androgen. It is expected that THG will also decrease gonadotropin secretion by the anterior pituitary gland as observed in our animal studies with the parent compound gestrinone [Kelly, P. A., J. Asselin, and F. Labrie, Endocrine regulation of growth and hormone receptor levels in DMBA-induced mammary tumors, in Steroids Receptors and the Management of Cancer, E. B. Thompson and M. E. Lippman, Editors. 1979, CRC Press Inc: Boca Raton, Fla. p. 3-29], thus leading to inhibition of testicular and ovarian activity in humans. In fact, in addition to its potent androgenic/anabolic and progestin [Death, A. K., et al., Tetrahydrogestrinone is a potent androgen and progestin. J Clin Endocrinol Metab, 2004. 89(5): p. 2498-500] activities, the toxicity profile of THG is completely unknown, thus making this compound an unknown risk for human use.

The power of gene-expression profiling has been clearly demonstrated in clinical medicine by the capability to divide adult acute myeloid leukemia into subgroups having different responses to specific treatments [Bullinger, L., et al., Use of gene-expression profiling to identify prognostic subclasses in adult acute myeloid leukemia. N Engl J Med, 2004. 350(16): p. 1605-16. Valk, P. J., et al., Prognostically useful gene-expression profiles in acute myeloid leukemia. N Engl J Med, 2004. 350(16): p. 1617-28.], thus facilitating choice of the best treatment for each category of cancer, while avoiding the serious side effects of inefficient treatments and the vital time lost trying inappropriate therapy while the cancer continues to progress [Mistry, A. R., et al., The molecular pathogenesis of acute promyelocytic leukaemia: implications for the clinical management of the disease. Blood Rev, 2003. 17(2): p. 71-97. Burnett, A. K., Current controversies: which patients with acute myeloid leukaemia should receive a bone marrow transplantation?—an adult treater's view. Br J Haematol, 2002. 118(2): p. 357-64].

Some methods for determination of the androgenic and anabolic activities are already known. The anabolic activity is an androgenic activity related to the constructive metabolism particularly referring to the muscle (enlargement in size). For example, the weight of prostate of castrated mammals, the growth of the size of ears and flank organs [Chen, C., A. Belanger, and F. Labrie, Adrenal steroid precursors exert potent androgenic action in the hamster sebaceous glands of flank organs and ears. Endocrinology, 1996. 137: p. 1752-1757] in the castrated hamster are widely used but these methods do not specifically distinguish the anabolic activities in muscle. The same limitation applies to in vitro methods using androgen-sensitive cell lines [Le Goff, J. M. and A. Belanger, Metabolism of tritiated C19 steroids by Shionogi mouse mammary tumors. Steroids, 1984. 44: p. 207-216. Belanger, C., R. Veilleux, and F. Labrie, Stimulatory effects of androgens, estrogens, progestins, and dexamethasone on the growth of the LNCaP human prostate cancer cells, in Steroid Formation, Degradation and Action in Peripheral, Normal, and Neoplastic Tissue, H. Bradlow, et al., Editors. 1990, Ann. New York Acad. Sci. p. 399-402.]. More specific to anabolic activity is the growth of an androgen-sensitive muscle, the levator ani, which has been recognized as a myotropic marker [Kelly, P. A., J. Asselin, and F. Labrie, Endocrine regulation of growth and hormone receptor levels in DMBA-induced mammary tumors, in Steroids Receptors and the Management of Cancer, E. B. Thompson and M. E. Lippman, Editors. 1979, CRC Press Inc: Boca Raton, Fla. p. 3-29: Death, A. K., et al., Tetrahydrogestrinone is a potent androgen and progestin. J Clin Endocrinol Metab, 2004. 89(5): p. 2498-500].

There is therefore a need in the art for a more precise and specific method to determine anabolic activity of compounds in muscles. Methods of the present invention are believed to address these needs.

### SUMMARY OF THE INVENTION

It is accordingly an object of the present invention to provide a method for determining the anabolic activity of a compound in the muscle.

It is another object to provide a method for determining what compounds should be considered inappropriate for use in athletic competition, or should be made illegal.

In one embodiment, the invention pertains to a method of determining the anabolic activity of a compound using a microarray technique which compares the in vivo changes of the genomic profile in a mammal induced by said compound versus the genomic profile induced by a known anabolic steroid, typically dihydrotestosterone (DHT).

In one embodiment, anabolic activity of a compound may be assessed by a method comprising the steps of
a) administering a suspected anabolic compound to a mammal;
b) extracting RNA of androgen-sensitive muscle tissues of said mammal;
c) converting said extracted RNA to cDNA;
d) transcribing said cDNA to produce RNA whose effects on androgen sensitive genes are evaluated;
e) comparing said effect to a corresponding effect with a known anabolic steroid.

It is preferred that the mammalian animal is a mouse. It is also preferred that the androgen-sensitive tissues are selected from the group consisting of levator ani and gastrocnemius muscles. In some embodiments, the androgen-sensitive tissue is collected and flash frozen prior to RNA extraction, in step (b) above.

In one embodiment, the known anabolic steroid is dihydrotestosterone (DHT).

The microarrays is a tool developed for large-scale analysis of gene expression, enabling the activities of hundreds of thousands of genes to be monitored simultaneously. The fundamental basis of DNA microarrays is the process of hybridization. Two strands of nucleic acid, DNA or RNA, hybridize if they are complementary to each other. This principle is exploited to measure the unknown quantity of one RNA molecule (target) on the basis of the amount of a complementary sequence (probe) that has hybridized to the target. Each probe sequence matches a particular messenger RNA present in the sample. The level of hybridization is usually quantified by measuring the level of a detectable fluorescent dye that can be detected by a light scanner that scans the surface of the chip. The concentration of a specific RNA messenger is a result of expression of its corresponding gene. Observing all the microarray spots at the same time gives the complete picture of the expression of all the genes represented on the microarray or gene expression profile. The GeneChip® Mouse Genome MOE 430 v2.0 Array represents over 34,000 well-characterized mouse genes. Each gene in the GeneChip is represented on average by 22 probes of 25 mer length each: 11 ‘perfect match’ (PM) probes that are complementary to the mRNA sequence, and 11 ‘mismatch’ (MM) probes that differ only by a single nucleotide at the central base. The MM value is utilized to adjust the PM intensity in order to incorporate some measure of non-specific cross-hybridization to mismatch probes. (a more detailed information about the GeneChips and the algorithms of analysis can by obtained in the web site: http://www.affymetrix.com).

Microarray experiments typically require 5-20 μg of total RNA per chip for sample labeling and hybridization. Nevertheless, very low amounts of total RNA are recovered from tissue biopsies, or other clinical samples. Linear amplification of RNA is them recommended by most manufacturers of commercially available microarrays. In the first steps, the RNA single strand is converted to DNA double strand. Synthetized DNA is them utilized to do a linear amplification using biotin-modified nucleotides. In this step, an enzyme, the T7 polymerase, use the dsDNA as a template to produce large amounts of biotinylated RNA. The enzymatic amplification technique is highly reproducible and maintains representation of the gene expression in the original sample

These methods are particularly suitable for selecting compounds whose anabolic activity make them appropriate for baning from use by athletes, or suitable to draw with a high degree of certainty a list of illegal or controlled compounds.

### BRIEF DESCRIPTION OF THE DRAWINGS

FIG. 1. shows the effect of increasing concentrations of methyltrienolone (R1881), testosterone (TESTO), tetrahydrogestrinone (THG) and dihydrotestosterone (DHT) on [³H]R1881 binding to the human androgen receptor. The incubation was performed with 3 nM [³H]R1881 for 16 h at 0-4° C. in the presence or absence of the indicated concentrations of unlabeled compounds.

FIG. 2. shows the effect of 7-day daily treatment with DHT or THG on prostate (A), seminal vesicle (B), preputial gland, (C) and muscle levator ani (D) weight, in gonadectomized (GDX)male C57BL6 mice. Data are expressed as the mean±SEM of 10 animals per group. **, p<0.01, experimental versus GDX-control mice, ++, experimental versus intact-control mice.

FIG. 3. shows the comparison of the effect of DHT on the gene expression profile by cluster analysis at 0.5, 1, 3, 6, 12 and 24 h following single subcutaneous injection of 0.1 mg of DHT or 0.5 mg of THG or 2, 3 and 7 days following daily administration of the same doses of the two steroids in the levator ani muscle (A), gastrocnemius muscle (B) or prostate (C) of mice GDX 7 days previously. The genes selected were those identified in common according to the Affymetrix, MAS 5.0 and RMA program [Gautier, L., et al., affy—analysis of Affymetrix GeneChip data at the probe level. Bioinformatics, 2004. 20(3): p. 307-15]. Color scale representing days of fold change due to treatments is shown below the figure.

FIG. 4 shows the effect of DHT and THG in mice skeletal muscle: Transcriptome changes in the highly androgen-responsive levator ani muscle. Clustering by gene and condition tree of 790 common genes for both treatments, DHT and THG (Condition tree; Similarity measure: Distance; Separation Ratio:1; Minimum Distance: 0.001. GeneSpring 7.2). Hierarchical clustering algorithm was applied to median normalized expression data of 790 predictive genes from 19 DHT and THG time points. The selected genes were clustered by Euclidean distance. Columns represents each one of the 790 selected gene and each row a particular treatement group. A pseudo-colored representation of relative intensity is shown such that red indicates high, blue low and yellow unchanged expression, with scale shown at the right.

### DETAILED DESCRIPTION OF THE INVENTION

We have taken advantage of the powerful technique of microarrays which can assess the level of expression of practically all genes in the genome to assess the in vivo changes of the genomic profile in the mouse, a species where 99% of the genes have direct counterparts in the human [Waterston, R. H., et al., Initial sequencing and comparative analysis of the mouse genome. Nature, 2002. 420(6915): p. 520-62]. Here we show that 790 and 1121 genes are modulated in common by THG and dihydrotestosterone (DHT), the most potent natural androgen and anabolic steroid, in the androgen-sensitive muscle levator ani and prostate, respectively, thus demonstrating without any doubt that THG is a highly potent anabolic steroid.

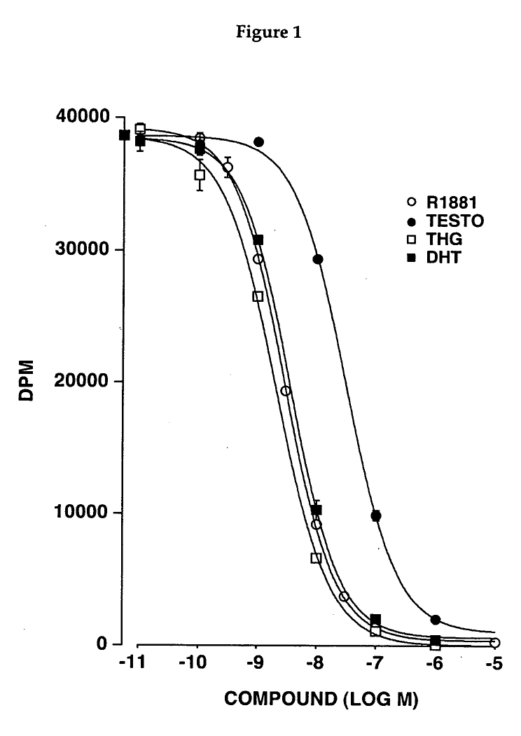

Since the first step in the action of androgens is binding to the androgen receptor (AR), we first compared the ability of THG, DHT, testosterone and methyltrienolone (R1881), to displace (³H) R1881 from the human AR. It can be seen in FIG. 1 that THG, R1881, DHT and testosterone have relative potencies of 1.0, 0.72, 0.58 and 0.07. These data already indicate the potential high androgenic activity of THG.

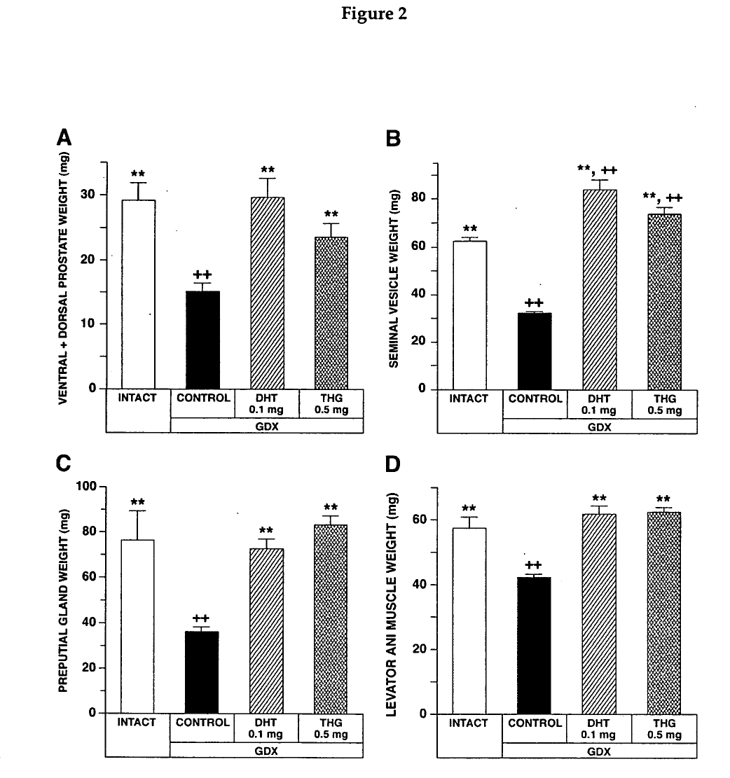

We next used the best recognized in vivo assay to assess the in vivo activity of THG [Labrie, C., A. Bélanger, and F. Labrie, Androgenic activity of dehydroepiandrosterone and androstenedione in the rat ventral prostate. Endocrinology, 1988. 123: p. 1412-1417]. In a preliminary experiment, THG has been found to be 20% as potent as DHT as stimulator of the weight of the mouse prostate, a most specific parameter of androgenic activity (data not shown). We could then select doses of the two compounds which maintain normal accessory sex organ weight following gonadectomy (GDX), namely 0.1 mg and 0.5 mg daily subcutaneous (s.c.)doses of DHT and THG, respectively. The daily injection of DHT completely reversed the GDX-induced atrophy of the prostate and led to a prostate weight similar to that of intact animals (FIG. 2A). Daily treatment with 0.5 mg of THG, on the other hand, reversed the effect of GDX, to a value not statistically different from intact controls. While GDX caused 48% (p<0.01) and 52% (p<0.01) decreases of seminal vesicle (FIG. 2B) and preputial gland (FIG. 2C) weights, respectively, the administration of DHT or THG completely reversed the GDX-induced atrophy of both tissues. Similar observations were made for the preputial gland. The levator ani is an androgen-sensitive muscle [Boissonneault, G., et al., Depressed translational activity in the androgen sensitive levator ani muscle of the rat. J Steroid Biochem, 1989. 32(4): p. 507-13] which has long been recognized as a myotropic marker of the androgenic/anabolic activity of steroids [Eisenberg, S., R. Buie, Jr., and L. Tobian, Jr., Adrenal cortical function in essential hypertension; a study of sweat sodium concentration. Am J Med Sci, 1950. 220(3): p. 287-9]. While GDX caused a 26% decrease in weight, the injection of DHT or THG increased weight of levator ani to values not different from intact animals.

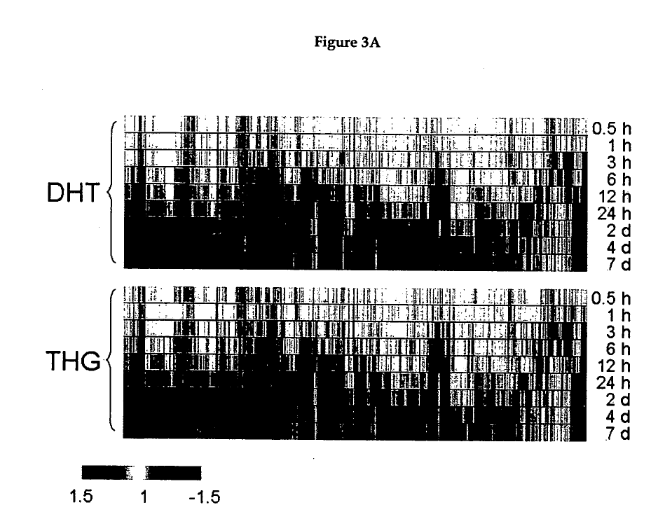

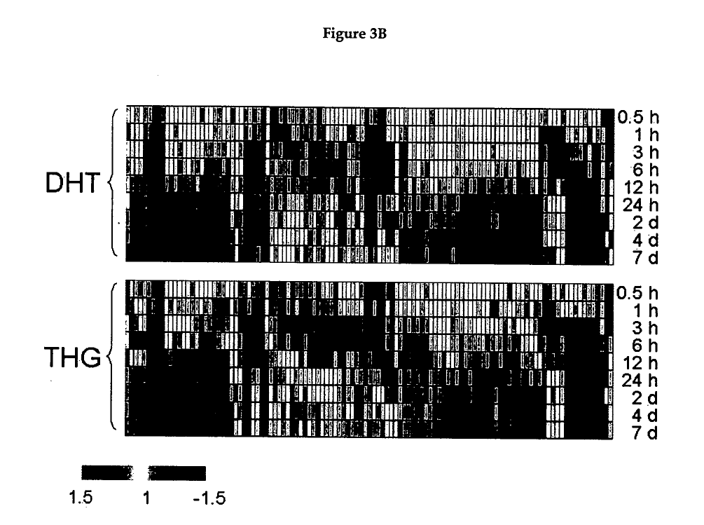

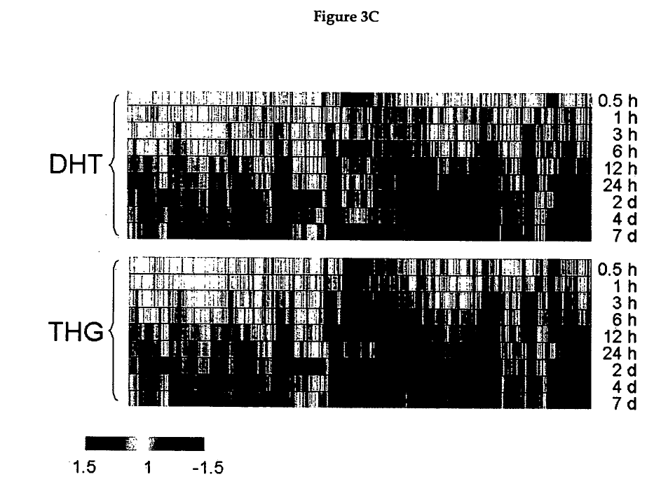

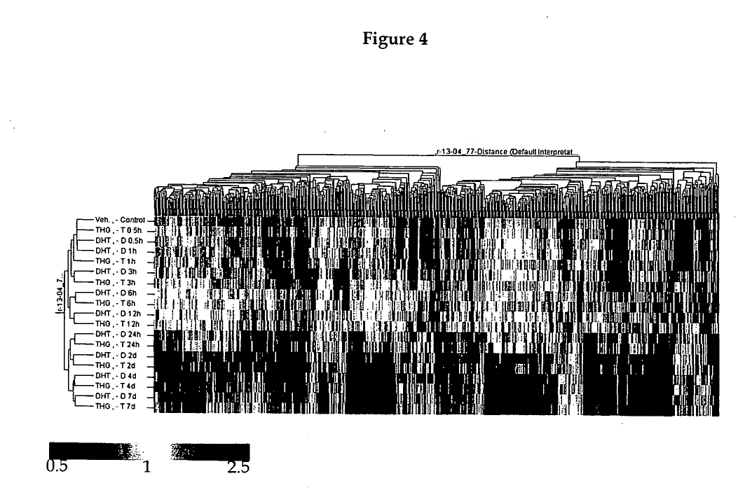

The potent androgenic activity of THG is best illustrated by the very close similarity of the pattern of genes up-(red) as well as down(blue)-regulated by DHT and THG in the androgen-sensitive levator ani muscle (FIG. 3A). In fact, the expression of 790 genes is commonly modulated by DHT and THG in the mouse levator ani. Although the gastroenemius muscle is less androgen-sensitive, FIG. 3B shows that 112 genes are commonly modulated by DHT and THG, thus resulting in another clear androgenic signature of THG in this tissue. In the prostate, on the other hand, the classical androgen-sensitive tissue [Labrie, C., A. Bélanger, and F. Labrie, Androgenic activity of dehydroepiandrosterone and androstenedione in the rat ventral prostate. Endocrinology, 1988. 123: p. 1412-1417], 1121 genes are commonly modulated by DHT and THG, thus clearly providing a typical androgenic signature to the action of THG (FIG. 3C). Not only a large number of genes are similarly up- or down-regulated in the three tissues by the two steroids but their time course of action is almost superimposable.

The extent of common gene modulation by the test compound, relative to the known anabolic comparison compound, (e.g. DHT, testosterone, testosterone esters, oxandrolone, fluoxymesterone or stanozolol) will vary among different test compounds. Ultimately, athletic or other authorities may determine the extent of common modulation that suggests that a compound be considered for regulatory restriction. The more the common modulation between the test compound and the anabolic comparison compound, the more reason for authorities to consider restrictive regulation of the test compound. For example, applicants suggest that when DHT is used as the anabolic comparison compound, common modulation of at least 60%, preferably at least 90%, be considered a threshold for restrictive regulation.

### EXAMPLE OF METHODS OF THE INVENTION

Materials and Methods

Animals

Eleven- to twelve-week-old male C57BL6 mice obtained from Harlan (Indianapolis, Ind.) were allowed to acclimate for 2 weeks. The animals were housed individually in an environmentally-controlled room (temperature: 22±3° C.; humidity: 50±20%; 12-h light-12-h dark cycles, lights on at 07:15 h). The mice had free access to tap water and a certified rodent feed (Lab Diet 5002 (pellet), Ralston Purina, St-Louis, Mo.). The experiment was conducted in an animal facility approved by the Canadian Council on Animal Care (CCAC) and the Association for Assessment and Accreditation of Laboratory Animal Care (AAALAC). The study was performed in accordance with the CCAC Guide for Care and Use of Experimental Animals.

Synthesis of THG

The synthesis of THG was performed by selective catalytic hydrogenation of gestrinone (H₂, Pd/C 10%, CH₂Cl₂, 1 atm, r.t., 1 h, 60% yield), in the medicinal chemistry division of our laboratory. The structure was confirmed by ¹H and ¹³C NMR and mass spectrometry. The purity of the compound was 98.9%.

Treatment

Animals weighing between 24.0 and 32.4 g (mean=28.2 g) were randomized according to body weight and were assigned to nineteen groups of 10 animals each. On day 1 of the study, animals were castrated (GDX) under isoflurane anesthesia. All animals were sacrificed 7 days after GDX. Mice were injected s.c. 0.5, 1, 3, 6, 12 or 24 h before sacrifice with DHT (0.1 mg/mouse) or THG (0.5 mg/mouse). DHT and THG suspended in 5% ethanol-0.4% methylcellulose, were injected subcutaneously. A GDX-vehicle injected group were used as a control. Eight intact mice of the same strain, age and body weight were sacrificed as described above and tissues were collected, weighed and discarded. The mice sacrificed 2, 4 and 7 days after starting treatment received daily injection of the steroids and were sacrificed 24 h after last injection under isoflurane anesthesia and exsanguinated via cardiac puncture. The prostate (ventral+dorsal), seminal vesicles, preputial glands as well as gastrocnemius and levator ani muscle were collected, freed from adhering tissue or fluid and weighed.

RNA extraction and microarrays tissues were snap-frozen in liquid nitrogen and kept at −80° C. prior to RNA extraction. Twenty micrograms of total RNA were converted to cDNA and transcribed in vitro to produce biotinylated cRNA that was hybridized to the MOE-430v2.0 GeneChip set (Affymetrix, Santa Clara, Calif.) according to the Affymetrix protocols. Scanned images were analyzed with Affymetrix GCOS v1.1 software and with GeneSpring 6.1 software (Silicon Genetics, Redwood City, Calif.) as described [Vasseur, S., et al., Gene expression profiling by DNA microarray analysis in mouse embryonicfibroblasts transformed by rasV12 mutated protein and the E1A oncogene. Mol Cancer, 2003. 2(1): p. 19].

Androgen Receptor (AR) Assay

Preparation of Human Embryonic Kidney (HEK-293) cells stably Transfected with Human AR (hAR): The pCMV neo-hAR plasmid [Huang, X.-F. and V. Luu-The, Modulation of the androgenic response by recombinant human 11-cis retinol dehydrogenase. J. Steroid Biochem., 2001. 77(2-3): p. 129-133] was transfected into HEK-293 cells using lipofectin transfection kit (Life Technologies, Ontario, Canada), and cells resistant to G418 were isolated as previously described [Dufort, I., et al., Characteristics of a highly labile human type 5 17beta-hydroxysteroid dehydrogenase. Endocrinology, 1999. 140(2): p. 568-574]. On the morning of the binding assay, a pellet of HEK-293 hAR cells was thawed, suspended in buffer, sonicated and centrifuged at 105 000×g for 90 min. The androgen binding assay was performed with the hydroxylapatite (HAP) method [Martel, C., et al., Binding characteristics of novel nonsteroidal antiestrogens to the rat uterine estrogen receptors. J. Steroid Biochem. Mol. Biol., 1998. 64: p. 199-205] using HEK-293 hAR cell cytosol preparation (0.1 ml) and 3 nM [³H]R1881.

The invention has been described in terms of preferred embodiments and examples, but is not limited thereby. Those of skill in the art will readily recognize the broader applicability and scope of the invention which is limited only by the patent claims that issue from this application or any patent application claiming priority (directly or indirectly) hereto.

What is claimed is:

1. A method of determining the anabolic activity of a compound in muscles using microarrays which permits to compare the in vivo changes of the genomic profile in the mammalian animal induced by said compound versus the genomic profile induced by a known anabolic steroid.

2. The method of claim 1 wherein the anabolic activity of a compound may be assessed by a method comprising the steps of
a) administering a suspected anabolic compound to a mammal;
b) extracting RNA of androgen-sensitive muscle tissues of said mammal;
c) converting said extracted RNA to cDNA;
d) transcribing said cDNA to produce RNA whose effects on androgen sensitive genes are evaluated;
e) comparing said effect to a corresponding effect with a known anabolic steroid.

3. The method of claim 2 wherein the mammalian animal is a mouse.

4. The method of claim 2 wherein the androgen-sensitive tissues are selected from the group consisting of levator ani and gastrocnemius muscles.

5. The method of claim 1 used for determining the anabolic activity of compounds administered to athletes.

6. The method of claim 2 wherein the known anabolic steroid is dihydrotestosterone (DHT).

7. The method of claim 2 wherein the androgen-sensitive muscle tissue is levator ani.

8. The method of claim 2 wherein the androgen-sensitive muscle tissue is gastrocnemius.
